(19) United States
(12) Patent Application Publication
(10) Pub. No.: US 2010/0053624 A1
(43) Pub. Date: Mar. 4, 2010

(54) BIOSENSOR

(76) Inventors: Kyung-Hwa Yoo, Seoul (KR); Donghyun Kim, Seoul (KR); Je Seung Oh, Gyeonggi-do (KR); Young Wook Chang, Seoul (KR); Seung Hwan Yoo, Incheon (KR)

Correspondence Address: FOLEY & LARDNER LLP, 150 EAST GILMAN STREET, P.O. BOX 1497, MADISON, WI, 53701-1497, US

(21) Appl. No.: 12/201,164

(22) Filed: Aug. 29, 2008

(51) Int. Cl. G01N 33/48; B05D 5/12; C23F 4/02; G01N 21/55; G01R 27/08

(52) U.S. Cl. 356/445; 427/58; 216/94; 324/722; 977/742

(57) ABSTRACT

A biosensor that can convert biological interactions into electrical and optical signals to sense a material to be analyzed is provided. The biosensor includes a substrate, a source electrode and a drain electrode formed on one surface of the substrate, a carbon nanotube connecting the source and drain electrodes, a metal gate covering the carbon nanotube, a recognition component immobilized on the metal gate, and a passivation layer covering the source and drain electrodes.

### TECHNICAL FIELD

This disclosure relates generally to biosensors.

### BACKGROUND

A number of biosensors have been proposed up to the present time. In general, a biosensor is a device in which a biological material configured to recognize a target biological material is coupled with a transducer that converts the biological interactions and recognition reactions into electrical or optical signals. A biosensor can analyze samples rapidly and precisely.

### SUMMARY

In one aspect, the disclosure provides a biosensor comprising a substrate; a source electrode and a drain electrode formed on one surface of the substrate; a carbon nanotube (CNT) configured to connect the source and drain electrodes; a metal gate configured to cover a portion of the CNT; a recognition component immobilized on the metal gate; and a passivation layer configured to cover the source and drain electrodes. A biosensor according to one embodiment is useful for sensing or detecting a target material by converting biomolecular interactions into corresponding output information and/or signals. The biosensor can be used for various purposes such as, but not limited to, drug screening, diagnosis of disease, monitoring of environmental contamination, food safety evaluation, etc.

### BRIEF DESCRIPTION OF THE DRAWINGS

FIG. 1 is a perspective view of an illustrative embodiment of a biosensor according to one embodiment of this disclosure.

FIG. 2 is a cross-sectional view taken along line A-A of FIG. 1.

FIG. 3 is a process diagram of an illustrative embodiment of a method of fabricating a biosensor according to one embodiment of this disclosure.

FIG. 4 is a process diagram of an illustrative embodiment of a process of immobilizing DNA to the biosensor shown in FIG. 1.

FIG. 5 is a cross-sectional view of an illustrative embodiment of a biosensor according to one embodiment of this disclosure.

FIG. 6 is a graphical illustration of electrical changes generated from a biosensor as a measure of DNA hybridization.

FIG. 7 is a graphical illustration of optical changes generated from a biosensor as a measure of DNA hybridization.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

A biosensor according to an embodiment of this disclosure can sense and/or detect a target material by converting biomolecular interactions into corresponding output information and/or signals, for example, electrical and optical signals. The biosensor can be used for various purposes such as, but not limited to, drug screening, diagnosis of disease, monitoring of environmental contamination, food safety evaluation, etc.

A biosensor according to one embodiment of this disclosure includes: a substrate; a source electrode and a drain electrode disposed on one surface of the substrate; a carbon nanotube (CNT) configured to electrically connect the source and drain electrodes; a metal gate covering a portion of the CNT; a recognition component immobilized on the metal gate; and a passivation layer covering the source and drain electrodes.

The substrate may be a material, such as but not limited to, for example, quartz or glass, and may be a semiconductor substrate doped with n- or p-type impurities. The surface area of the substrate 101 may be larger than 100 μm by 100 μm, and the thickness of the substrate may be approximately 100 μm to 600 μm, approximately 200 μm to 500 μm, approximately 300 μm to 400 μm.

After at least two transition metal catalysts such as, but not limited to, cobalt or iron are patterned on one surface of the substrate by photolithography and lift-off, each catalyst is capable of growing the CNT. At least one CNT forms between, with its two ends rooted in, the two opposing catalysts. The source electrode and the drain electrode are then formed to fully cover the catalysts. The CNT is electrically connected to the source electrode and the drain electrode (one at each end of the CNT), and acts as a channel for transporting charges between the source electrode and the drain electrode. That is, the CNT may be used as a channel of a field effect transistor (FET). The CNT may be a single-walled CNT, a double-walled CNT or a multi-walled CNT, and has the properties of a semiconductor. In a case the CNT is a single-walled CNT, the diameter of the CNT may be 2 to 3 nm.

The source and drain electrodes may be formed of any material having electrical conductivity, for example, gold (Au), platinum (Pt) or palladium (Pd). In addition, each of the electrodes may have a single-layered structure, or a multi-layered structure such as, but not limited to, titanium (Ti)/Au, chromium (Cr)/Au, Ti/Pt, Cr/Pt, Ti/Pd, Cr/Pd, etc. A multi-layered structure electrode may be formed by depositing a layer of Ti or Cr on the substrate and then depositing a layer of Au, Pt or Pd thereon.

The metal gate covers a portion of the CNT. The metal gate may be formed of a material having a superior electrical conductivity such as, but not limited to, Au or silver (Ag). In addition, Cr, Ti, aluminum (Al) or calcium (Ca) may be used as an adhesive layer to fabricate sensors having different properties.

The recognition component may be a large variety of biomolecules such as, but not limited to, oligonucleotide, aptamers, receptors, antibodies, etc. In one embodiment, the recognition component may be a single-stranded oligonucleotide such as DNA or RNA. The single-stranded oligonucleotide may be a thiol-modified oligonucleotide. In the case of DNA, the biosensor has a recognition DNA (RD, recognition component) immobilized on the surface of the metal gate. Electrical and/or optical signals are generated as a result of hybridization between the RD and a target DNA (TD, target component).

The passivation layer covers the source electrode and the drain electrode, and may be formed of a silicon oxide (SiOₓ) such as SiO₂ or a polymer such as but not limited to polymethyl methacrylate (PMMA), polyester, polystyrene, polyethylene terephthalate (PET), polycarbonate (PC), polyvinylidene chloride or triacetate (TAC). The metal gate, unlike the source electrode and the drain electrode, is exposed and not covered by the passivation layer. The passivation layer covers a portion of contact between the source electrode and the CNT and a portion of contact between the drain electrode and the CNT. The thickness of the passivation layer may be approximately 10 nm to 100 nm, approximately 30 nm to 80 nm, approximately 50 nm to 60 nm.

A biosensor according to an embodiment of this disclosure and a method of fabricating the same will now be described with reference to the accompanying drawings. The drawings are provided for simplicity of description, and the thickness of layers, shapes of electrodes and members may be exaggerated or reduced for clarity.

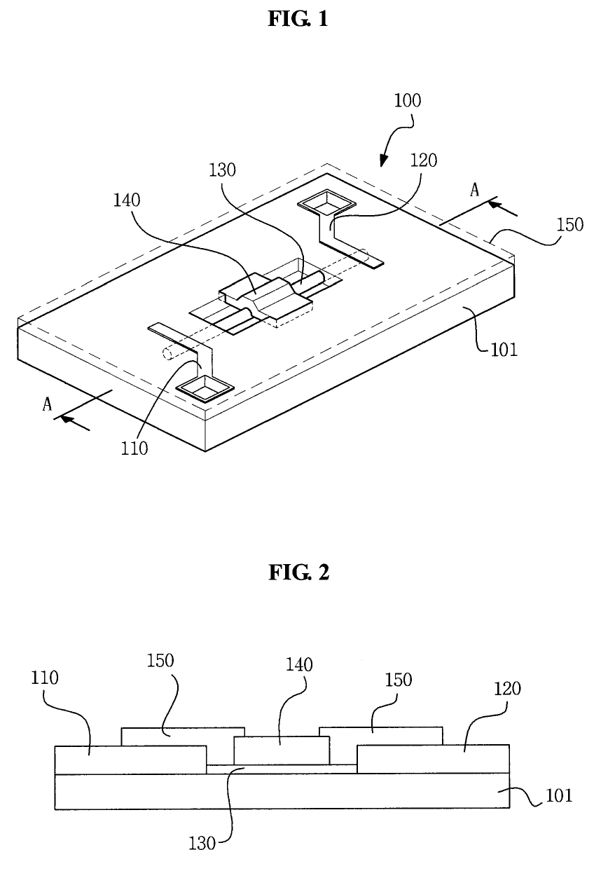

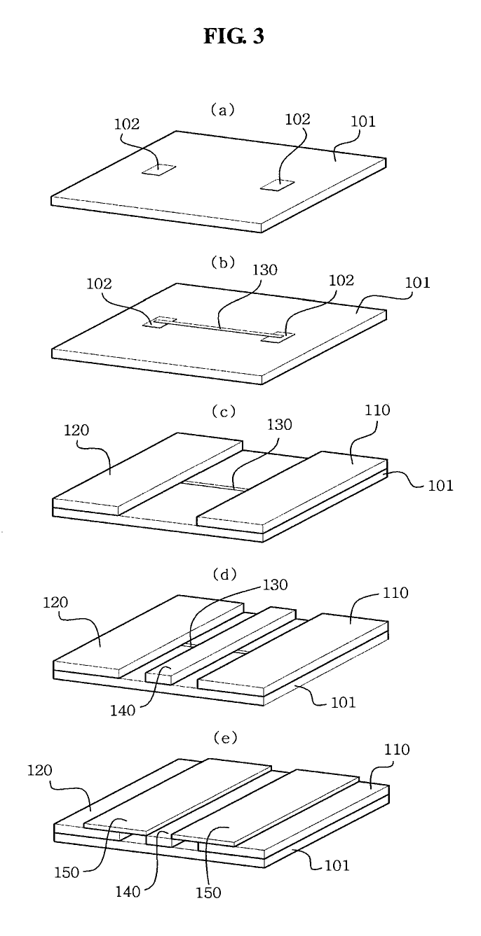

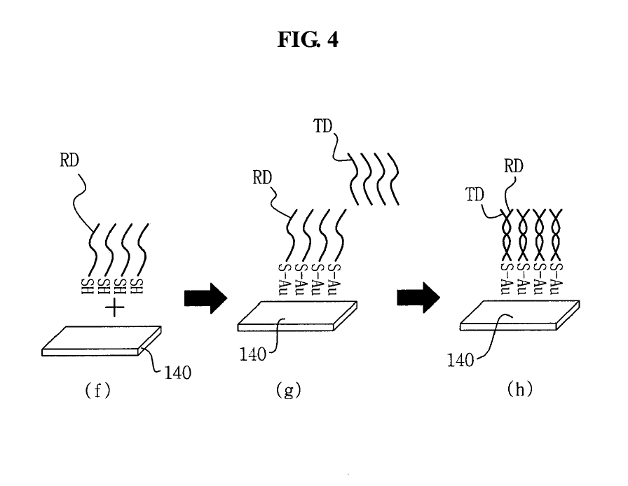

FIG. 1 is a perspective view of a biosensor. FIG. 2 is a cross-sectional view taken along line A-A of FIG. 1. FIG. 3 is a process diagram illustrating a method of fabricating the biosensor shown in FIG. 1.

As shown in FIGS. 1 to 3, a biosensor 100 includes a substrate 101, a source electrode 110, a drain electrode 120, a CNT 130, a metal gate 140, a recognition component immobilized on the metal gate and a passivation layer 150.

The substrate 101 may be formed of a transparent material, such as but not limited to, quartz or glass, and may be a semiconductor substrate doped with n- or p-type impurities. The surface area of the substrate 101 may be larger than 100 μm by 100 μm, and the thickness of the substrate may be 100 μm to 600 μm, approximately 200 μm to 500 μm, approximately 300 μm to 400 μm.

After at least two transition metal catalysts 102 such as, but not limited to, cobalt or iron are patterned on one surface of the substrate 101 by photolithography and lift-off, each catalyst is capable of growing the CNT 130. At least one CNT 130 forms between, with its two ends rooted in, the two opposing catalysts 102. The source electrode 110 and the drain electrode 120 are then formed to fully cover the catalysts 102. The CNT 130 is electrically connected to the source electrode 110 and the drain electrode 120 (one at each end of the CNT), and acts as a channel for transporting charges between the source electrode 110 and the drain electrode 120. That is, the CNT 130 may be used as a channel of a field effect transistor (FET). The CNT 130 may be a single-walled CNT, a double-walled CNT or a multi-walled CNT, and has the properties of a semiconductor. In case the CNT is a single-walled CNT, the diameter of the CNT may be 2 nm to 3 nm.

The source electrode 110 and the drain electrode 120 are formed on one surface of the substrate 101, as illustrated FIG. 3. The source electrode 110 and the drain electrode 120 may be formed of any material having a superior electrical conductivity, for example, gold (Au), platinum (Pt) or palladium (Pd). In addition, each of the electrodes 110 and 120 may have a single-layered structure, or a multi-layered structure such as, but not limited to, titanium (Ti)/Au, chromium (Cr)/Au, Ti/Pt, Cr/Pt, Ti/Pd, Cr/Pd, etc. An electrode having such a multi-layered structure may be formed by first depositing Ti or Cr on the substrate 101 and then depositing Au, Pt or Pd thereon.

The CNT 130 is configured to electrically couple to the source electrode 110 and the drain electrode 120 at both ends thereof, and acts as a channel for transporting charges between the source electrode 110 and the drain electrode 120. That is, the CNT 130 may be used as a channel of a field effect transistor (FET). In case the CNT is a single-walled CNT, the diameter of the CNT may be 2 nm to 3 nm.

The metal gate 140 covers a portion of the CNT 130, and is formed in a space between the source electrode 110 and the drain electrode 120. Such a metal gate 140 may be formed of a material having a superior electrical conductivity, such as but not limited to, Au or silver (Ag). In addition, Cr, Ti, aluminum (Al) or calcium (Ca) may be used as an adhesive layer. The metal gate 140 having such an adhesive layer may be formed by first depositing Cr, Ti, Al or Ca and then depositing Au, Pt or Pd thereon.

The passivation layer 150 covers the source electrode 110 and the drain electrode 120, and may be formed of a silicon oxide (SiOₓ) such as SiO₂ or a synthetic resin such as, but not limited to, polymethyl methacrylate (PMMA), polyester, polystyrene, polyethylene terephthalate (PET), polycarbonate (PC), polyvinylidene chloride or triacetate (TAC). The metal gate 140, unlike the source electrode 110 and the drain electrode 120, is exposed and not covered by the passivation layer 150. The passivation layer 150 can cover a portion of contact between the source electrode and the CNT and a portion of contact between the drain electrode and the CNT. The thickness of the passivation layer may be 10 nm to 100 nm.

As shown in FIG. 4, the recognition component (e.g., a single-stranded DNA) is immobilized on the surface of the metal gate 140. When the metal gate 140 is formed of Au, the single-stranded DNA on the surface of the metal gate 140 is a thiol-modified DNA, and the thiol connected to its end may be attached to the Au or Ag surface by means of self-assembly (step f). A method of fabricating the biosensor having the above structure will now be described in detail with reference to FIG. 3. Transition metal catalysts 102 such as, but not limited to, cobalt or iron are patterned on one surface of the substrate 101 by photolithography and lift-off.

A CNT 130 is grown between the catalyst patterns by a chemical vapor deposition (CVD) method in a gaseous atmosphere containing methane and hydrogen.

The source and drain electrodes 110 and 120 are positioned to be electrically connected to each other through the CNT 130. Each of the electrodes 110 and 120 may be a Cr (or Ti)/Au, Cr (or Ti)/Pt, or Cr (or Ti)/Pd electrode, and may be deposited on one surface of the substrate 101 using photolithography or a metal mask. In addition, the thickness of each layer of the Cr (or Ti)/Au, Cr (or Ti)/Pt, or Cr (or Ti)/Pd electrode may be 2 nm to 5 nm (in the case of Cr or Ti), and 50 nm to 150 nm (in the case of Au, Pt, or Pd).

A metal gate 140 is formed over a portion of the CNT 130, and may be formed between the source electrode 110 and the drain electrode 120 (step d). Some portion of the metal gate 140 is disposed on the CNT 130, and the remaining portion of the metal gate 140 is disposed on the substrate 101, as shown in FIG. 4. The metal gate 140 may be formed of Au or Ag as described above, and may be formed by first depositing Cr, Ti, Al or Ca (as an adhesive layer) and then depositing Au thereon in the case of a metal gate having a multi-layered structure. The thickness of each layer may be 2 to 5 nm (in the case of Cr, Ti, Al or Ca), and 40 to 50 nm (in the case of Au). The deposition may be carried out by photolithography, E-beam lithography or a metal mask, and the like.

A passivation layer 150 is formed so as to cover the source electrode 110 and the drain electrode 120, The passivation layer covers a portion of contact between the source electrode and the CNT and a portion of contact between the drain electrode and the CNT. As described above, the passivation layer 150 may be formed by the same method as the method of forming the electrodes using a silicon oxide (SiOₓ) such as SiO₂ or a synthetic resin such as PMMA, polyester, polystyrene, polyethylene terephthalate (PET), polycarbonate (PC), polyvinylidene chloride or triacetate (TAC).

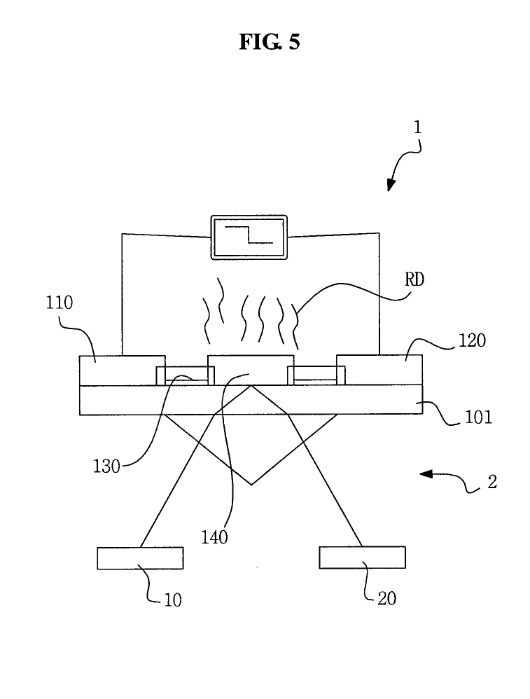

FIGS. 4 and 5 are cross-sectional views illustrating a biosensor 100 according to an embodiment of this disclosure. The biosensor 100 has a recognition DNA (RD) immobilized on the surface of the metal gate 140, and analyzes electrical and optical signals resulting from hybridization between the RD and a target DNA (TD) to sense the TD.

Referring to FIG. 4, a method of immobilizing the RD on the surface of the metal gate 140 will now be described. The biosensor is first immersed into a thiolated single-stranded DNA solution for about 2 to 10 hours. The RD with known nucleotide sequence and with a concentration of equal to or greater than 10 μM in 1 M phosphate buffer pH 7.0 with 1 M NaCl can be immobilized to the metal gate surface by self-assembly. The biosensor is washed with distilled water several times, and then dried using a gas such as, but not limited to, nitrogen or argon (step f). In a case the recognition component is a single-stranded DNA, the above drying step can be omitted. The hybridization can be preformed according to the nature of the DNA, its size, and so on, and the hybridization can be conducted by a general protocol (steps g and h). This hybridization can be performed by any technique known to the person skilled in the art.

Referring to FIG. 5, the biosensor according to an embodiment of this disclosure includes an electrical signal converting unit 1 and an optical signal converting unit 2.

The electrical signal converting unit 1 includes a source electrode 110, a drain electrode 120, a CNT 130 configured to electrically connect the source electrode 110 to the drain electrode 120, and a metal gate 140. This structure acts as a metal-semiconductor FET (MES-FET), that is, the CNT 130 is used as a channel of the FET. When a single-stranded DNA (i.e., RD) hybridizes with a TD to become a double-stranded DNA, their hybridization changes the charge state of the metal gate 140 surface. Because the metal gate 140 is associated with the CNT 130 for controlling the conductance of the CNT 130, the charge density of the CNT 130 is changed, and the change in conductance is measured to sense the TD. The biosensor of this disclosure can detect the TD in the range of 10 pico M to 100 micro M. The DNA hybridization occurs when the base sequences are complementary or nearly so. As described above, the hybridization can be preformed according to the nature of the DNA, its size, and so on, and the hybridization can be conducted by a general protocol. This hybridization can be performed by any technique known to the person skilled in the art.

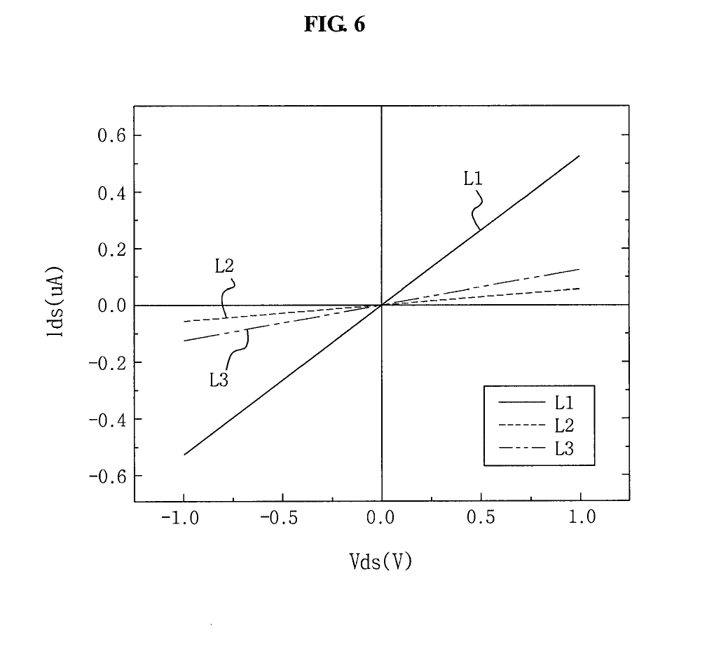

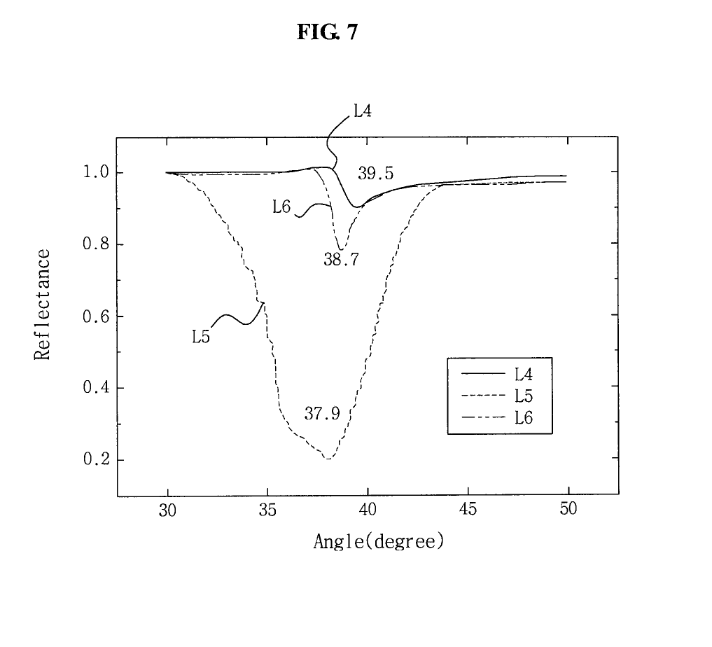

The optical signal converting unit 2 includes a substrate 101 formed for example, of quartz or glass and a metal gate 140. The optical signal converting unit 2 operates according to the principle of surface plasmon resonance (SPR). Thus, the biosensor can convert a fine change in resonance angle of the metal gate into an optical signal and carry out measurements while the complementary hybridization of the DNA occurs on the surface of the metal gate 140. For example, a laser beam irradiated from an external light source 10 will penetrate the substrate 101 and be reflected by the metal gate 140, and the reflected laser beam will penetrate the substrate 101 again to be detected by an external detector 20. The biosensor measures the change of resonance angle caused by the rapidly changed reflectance. FIG. 6 is a graph illustrating electrical changes measured from the biosensor according to the complementary hybridization of DNA, and FIG. 7 is a graph illustrating optical changes measured from the biosensor according to the complementary hybridization of DNA. Referring to FIG. 6, it can be seen that the resistance of the biosensor is changed while the single-stranded DNA is immobilized and is complementarily hybridized to form the double-stranded DNA, and SPR angle shifts can be simultaneously measured to sense the complementary hybridization of DNA. The RD with sequence 5′-/5ThilMC6-D/CAAACGGTACTAGACGCGTATAACTGACTT-3′ (SEQ ID NO: 1) was used in a concentration of 30 μM in 1 M phosphate buffer pH 7.0 with 1 M NaCl. After immobilizing the RD on the metal gate, the TD with sequence 5′AAGTCAGTTATACGCGTCTAGTACTTG-3′ (SEQ ID NO: 2) was used in a concentration of 10 pico M in the same phosphate buffer.

Referring to FIG. 6, L1 denotes the basic conductivity between the source and drain electrodes of the biosensor, L2 denotes the electrical conductivity between the source and drain electrodes after the RD is immobilized on the metal gate 140, and L3 denotes the electrical conductivity between the source and drain electrodes after the RD and the TD are complementarily hybridized in the metal gate 140 of the biosensor.

Referring to FIG. 7, L4 denotes a basic SPR signal of the metal (Au) in the biosensor, L5 denotes an SPR signal after the RD is immobilized on the metal (Au) in the biosensor, and L6 denotes an SPR signal after the RD and the TD are complementarily hybridized on the metal (Au) of the biosensor. The values of FIG. 7 denote the angles causing resonance to occur.

According to an embodiment of this disclosure, the biosensor can sense an electrical signal by being configured as a field effect transistor (FET) and an optical signal through a surface plasmon resonance (SPR) technique. The biosensor simultaneously converts biological interactions into electrical and optical signals to monitor the complementary hybridization of DNA, so the biosensor has high sensitivity and reliability. Also, the biosensor of this disclosure can sense a wide range of biomolecules such as, but not limited to, oligonucleotide, aptamers, receptors, antibodies, etc.

### EQUIVALENTS

The biosensor and method of fabricating the same as described above may be applied to various biosensors and methods of fabricating the same without departing from the scope of the claims.

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A biosensor comprising:
a substrate;
a source electrode and a drain electrode formed on one surface of the substrate;
a carbon nanotube (CNT) configured to connect the source and drain electrodes;
a metal gate configured to cover a portion of the CNT;
a recognition component immobilized on the metal gate; and
a passivation layer configured to cover the source and drain electrodes.

2. The biosensor according to claim 1, wherein the metal gate is formed of gold (Au) or silver (Ag).

3. The biosensor according to claim 1, further comprising: an adhesive layer disposed between the metal gate and the CNT.

4. The biosensor according to claim 3, wherein the adhesive layer is formed of chromium (Cr), titanium (Ti), aluminum (Al) or calcium (Ca).

5. The biosensor according to claim 1, wherein the substrate is formed of transparent quartz or glass.

6. The biosensor according to claim 1, wherein each of the electrodes is formed of gold (Au), platinum (Pt) or palladium (Pd).

7. The biosensor according to claim 1, wherein each of the electrodes has a multi-layered structure and the multi-layered structure includes Ti/Au, Cr/Au, Ti/Pt, Cr/Pt, Ti/Pd, or Cr/Pd.

8. The biosensor according to claim 1, wherein a portion of the metal gate is exposed from the passivation layer.

9. The biosensor according to claim 1, wherein the passivation layer is formed of a silicon oxide or a polymer.

10. The biosensor according to claim 1, wherein the recognition component is a single-stranded oligonucleotide.

11. The biosensor according to claim 10, wherein the oligonucleotide is a thiol-modified oligonucleotide.

12. A method of fabricating a biosensor, comprising:
growing a carbon nanotube (CNT) on one surface of a substrate;
forming a source electrode and a drain electrode on one surface of the substrate and configured so as to be electrically connected to each other through the CNT;
forming a metal gate that covers a portion of the CNT;
immobilizing a recognition component on the metal gate; and
forming a passivation layer that covers the source and drain electrodes.

13. The method according to claim 12, wherein the metal gate is formed on an adhesive layer after the adhesive layer is formed on the CNT.

14. The method according to claim 13, wherein each of the electrodes is formed by photolithography or a metal mask.

15. The method according to claim 13, wherein the metal gate is formed by photolithography, E-beam lithography, or a metal mask.

16. The method of sensing a target component using a biosensor according to claim 1 comprising:
immobilizing the recognition component on the surface of the metal strip;
performing measurements of conductance changes of the CNT; and
performing measurements of SPR angle shifts on the metal gate.
